(19) United States
(12) Patent Application Publication
(10) Pub. No.: US 2023/0245737 A1
(43) Pub. Date: Aug. 3, 2023

(54) MEDICAL INFORMATION MANAGEMENT APPARATUS AND STORAGE MEDIUM

(71) Applicant: Konica Minolta, Inc., Tokyo (JP)

(72) Inventor: Youko KOUDA, Tokyo (JP)

(21) Appl. No.: 18/159,458

(22) Filed: Jan. 25, 2023

(30) Foreign Application Priority Data
Feb. 3, 2022 (JP) 2022-015417

(51) Int. Cl. G16H 10/60

(52) U.S. Cl. CPC G16H 10/60

(57) ABSTRACT

A medical information management apparatus includes a hardware processor. The hardware processor obtains first medical information that is input through a patient terminal used by a patient and that includes vital information of the patient; attaches note information to the first medical information being displayed on a display in accordance with a manipulation of an operation receiver by a healthcare practitioner; and outputs the first medical information to which the note information is attached.

### CROSS-REFERENCE TO RELATED APPLICATIONS

The present invention claims priority under 35 U.S.C. § 119 to Japanese Application, 2022-015417, filed on Feb. 3, 2022, the entire contents of which being incorporated herein by reference.

### TECHNICAL FIELD

The present disclosure relates to a medical information management apparatus and a storage medium.

### DESCRIPTION OF THE RELATED ART

When a patient suffering from diabetes, hypertension, or the like consults a doctor, the patient provides the medical facility with his/her information by verbally conveying vital information that the patient has recorded daily or by showing notes of the vital information to the doctor. The patient may receive advice about the vital information from the medical facility. However, the patient may leave the consultation room without clearly understanding the advice, or the patient may not be able to remember details about the diagnosis later.

JP2008-33779A proposes a healthcare management support system that allows a user to manage his/her daily healthcare without visiting a medical facility. According to the system, the user sends vital information from the user terminal to a central server; the medical facility refers to the vital information in the central server; and the medical facility sends advice about the healthcare to the user by email.

### SUMMARY OF THE INVENTION

According to the above known system, the patient sends and registers the vital information to the central server, and in response, the doctor sends advice to the patient by a separate email. The patient receives and checks the email but may not grasp at a glance what kind of comments is made to the vital information sent by the patient. Thus, the above known system has a problem about viewability.

The present invention has been conceived in view of the above issue. An object of the present invention is to present information input by healthcare practitioners such that the information is easily understood, the information being with respect to medical information that includes vital information input by the patient.

To achieve at least one of the abovementioned objects, according to an aspect of the present invention, a medical information management apparatus includes a hardware processor that: obtains first medical information that is input through a patient terminal used by a patient and that includes vital information of the patient; attaches note information to the first medical information being displayed on a display in accordance with a manipulation of an operation receiver by a healthcare practitioner; and outputs the first medical information to which the note information is attached.

According to another aspect of the present invention, a non-transitory computer-readable storage medium stores a program that causes a computer to perform: obtaining first medical information that is input through a patient terminal used by a patient and that includes vital information of the patient; attaching note information to the first medical information being displayed on a display in accordance with a manipulation of an operation receiver by a healthcare practitioner; and outputting the first medical information to which the note information is attached.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows a structure of a medical information management system in a first embodiment of the present invention.

FIG. 2 shows a ladder chart of a process to be performed by the medical information management system in the first embodiment.

FIG. 3 shows an example of medical care guidance window that is displayed on a doctor terminal.

FIG. 4 shows an example of the medical care guidance window in which note information is attached.

FIG. 5 shows a structure of a medical information management system in a second embodiment of the present invention.

FIG. 6 shows a ladder chart of a process to be performed by the medical information management system in the second embodiment.

FIG. 7 shows the ladder chart of the process to be performed by the medical information management system in the second embodiment.

FIG. 8 shows an example of the medical care guidance window in which note information is attached to first medical information, second medical information, and third medical information.

FIG. 9 shows an example of an electrocardiogram window in which electrocardiogram data is enlarged.

FIG. 10 shows an example of a blood pressure classification table window in which a blood pressure classification table is enlarged.

### DETAILED DESCRIPTION

Hereinafter, embodiments of a medical information management apparatus according to the present invention will be described with reference to the figures. However, the scope of the invention is not limited to the illustrated examples.

### First Embodiment

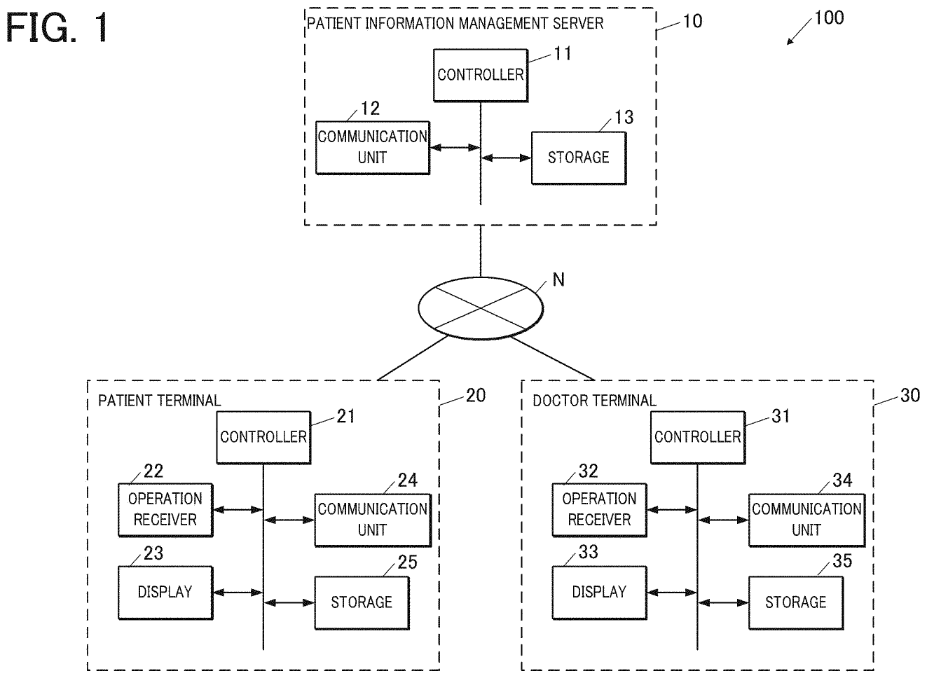

(Configuration of medical information management system) FIG. 1 shows a system configuration of a medical information management system 100 in a first embodiment. As shown in FIG. 1, the medical information management system 100 includes a patient information management server 10; a patient terminal 20 to be used by a patient; and a doctor terminal 30 (medical information management apparatus) to be used by a doctor in a medical facility. The patient information management server 10, the patient terminal 20, and the doctor terminal 30 are connected to exchange data over a communication network N (e.g., internet). The number of patient terminals 20 and the number of doctor terminals 30 is not specifically limited.

The patient information management server 10 accumulates and manages medical information on patients registered by the patient terminal 20 and the doctor terminal 30. The patient information management server 10 may be a cloud server established in a cloud environment.

The patient terminal 20 is a terminal device used by a patient, such as a smartphone.

The doctor terminal 30 is a computer device such as a personal computer (PC) or a tablet terminal that is used by a doctor (healthcare practitioner) who belongs to the medical facility.

The patient terminal 20 and the doctor terminal 30 are used when registering patients' medical information to the patient information management server 10 and when referring to patients' medical information that is managed by the patient information management server 10, for example.

For example, a patient inputs first medical information by using the patient terminal 20 at home and sends the first medical information to the patient information management server 10. The first medical information includes vital information, exercise information, medication information, meal information, and feeling information, for example.

A doctor checks the first medical information stored in the patient information management server 10 by using the doctor terminal 30 before or while seeing the patient.

During examination of the patient, the doctor also provides medical care guidance to the patient while showing the first medical information to the patient and writing notes in the first medical information on the doctor terminal 30. The doctor terminal 30 attaches note information (notes written to the first medical information) to the first medical information and sends the information to the patient information management server 10. The note information includes information for pointing portions to be attended (e.g., figures, arrows); explanations of contents/tendency in medical information; and/or supplementary explanations.

The patient looks at the information stored in the patient information management server 10 by using the patient terminal 20 at home.

The patient information management server 10 provides the patient terminal 20 with (i) the first medical information of the patient corresponding to the patient terminal 20 and (ii) the note information attached by the doctor's operation.

(Configuration of Patient Information Management Server)

As shown in FIG. 1, the patient information management server 10 includes a controller 11, a communication unit 12, and a storage 13. These components are connected via a bus.

The controller 111 includes a central processing unit (CPU) and a random access memory (RAM), and centrally controls operations of the components of the patient information management server 10. More specifically, the CPU of the controller 11 reads various processing programs stored in the storage 13, loads them into the RAM, and performs various processes in cooperation with the programs.

The communication unit 12 includes a network interface. The communication unit 35 sends and receives data to and from external apparatuses connected over the communication network N.

The storage 13 may consist of a hard disk drive (HDD) and/or a solid state drive (SSD), for example. The storage 13 stores programs of various processes and parameters and files necessary for executing the programs. For example, the storage 13 stores: a Web server program for performing functions of a Web server that communicates with Web browsers installed in external apparatuses in accordance with the HTTP protocol and that provides the Web browsers with various Web windows; and application programs to be operated on the Web server.

The storage 13 stores patient information, medical facility information, and healthcare practitioner information.

The patient information refers to information on each patient. The patient information includes a patient's ID, name, address, phone number, e-mail address, and the name of a medical facility being used by the patient.

The medical facility information refers to information on each medical facility. The medical facility information includes the name, address, and phone number of a medical facility.

The healthcare practitioner information refers to information on each healthcare practitioner. The healthcare practitioner information includes a healthcare practitioner's name, phone number, e-mail address, and the name of the medical facility to which the healthcare practitioner belongs.

The storage 13 also stores first medical information in association with the patient information. The first medical information is data input by the patient terminal 20. Each data item of the first medical information is associated with the measurement date and time, input date and time, obtained date and time, and target date and time. The first medical information includes vital information, exercise information, medication information, meal information, and feeling information.

The vital information includes blood pressure, a heart rate, a body temperature, a blood sugar level, a weight, and SpO2 (saturation of peripheral oxygen in artery).

The exercise information includes the number of steps, the moved distance, and information on the exercise done by the patient (the type, the number of times, and duration of the exercise).

The medication information includes information on a medicine(s) that was actually taken by the patient. The medication information includes information on whether the patient took the medicine; the type of medicine taken; the date and time the patient took the medicine; the intervals between doses; and the amount of medicine.

The meal information includes images of meals that the patient had; contents of meals; nutrients; and caloric intakes.

The feeling information includes information on the patient's feeling (good, normal, bad).

The first medical information may include body regions that ache or that become numb; and symptoms such as dizziness, sleeplessness, and diarrhea.

The storage 13 also stores image data of a medical care guidance window in association with the patient information. The image data of the medical care guidance window is generated by the doctor terminal 30 and includes a layer containing the first medical information and a layer containing the note information. When the patient looks at the medical care guidance window on the patient terminal 20, the note information is superposed on the first medical information and displayed.

The controller 11 allows a doctor terminal 30 to access information associated with patient information, when the patient information includes the name of the medical facility corresponding to the doctor terminal 30.

The controller 11 allows a patient terminal 20 to access information associated with patient information, when the patient information is on the patient corresponding to the patient terminal 20.

(Configuration of Patient Terminal)

As shown in FIG. 1, the patient terminal 20 includes a controller 21, an operation receiver 22, a display 23, a communication unit 24, and a storage 25. These components are connected via a bus.

The controller 21 includes a CPU and a RAM and centrally controls processing operations of the components of the patient terminal 20. More specifically, in accordance with operation signals input by the operation receiver 22 or instruction signals received by the communication unit 24, the CPU of the controller 21 reads out various processing programs stored in the storage 25, loads them into the RAM, and executes various processes in cooperation with the programs.

The operation receiver 22 includes operation keys such as a power key for turning on/off power; and a touchscreen laid on the display 23. The operation receiver 22 outputs, to the controller 21, operation signals corresponding to manipulated operation keys and operation signals corresponding to positions touched by the operator's finger.

The display 23 includes a liquid crystal display (LCD), for example. The display 23 displays various windows/screens in accordance with instructions of display signals input by the controller 21.

The communication unit 24 includes a network interface, for example. The communication unit 24 sends and receives data to and from external apparatuses connected over the communication network N. The communication unit 24 also performs wireless communications by using mobile phone lines, and sends and receives data to and from external apparatuses connected over the communication network N. The communication unit 24 also performs wireless communications by using the near field communication (NFC) and/or Bluetooth (registered trademark) and sends and receives data to and from external apparatuses (e.g., a vital measurement apparatus).

The storage 25 consists of a HDD and/or a nonvolatile semiconductor memory, for example. The storage 25 stores various processing programs and parameters and files necessary for executing the programs. For example, the storage 25 stores a Web browser program for performing functions of a Web browser and various application programs.

The controller 21 sends first medical information that has been input by the patient terminal 20 (e.g., vital information, exercise information, medication information, meal information, feeling information) to the patient information management server 10 via the communication unit 24. Herein, “input” includes inputs with the operation receiver 22, reception of data via the communication unit 24, and reading of data from storage media, for example. The first medical information to be sent from the patient terminal 20 to the patient information management server 10 is associated with the measurement date and time, input date and time, obtained date and time, and target date and time.

For example, the controller 21 obtains vital information from the vital measurement apparatus via the communication unit 24. The controller 21 may obtain vital information that is input by the patient with the operation receiver 22.

The controller 21 calculates the number of steps and the traveled distance on the basis of changes in the acceleration rate that are obtained by an acceleration sensor installed in the patient terminal 20. The controller 21 obtains information on the exercise done by the patient (e.g., the type, the number of times, the duration of the exercise) that is input by the patient with the operation receiver 22.

The controller 21 obtains the medication information and feeling information input by the patient with the operation receiver 22.

The controller 21 obtains image data of meal images that is generated by a camera mounted on the patient terminal 20. The controller 21 may obtain meal images from an external digital camera via the communication unit 24. The controller 21 may obtain the meal contents, nutrients, and caloric intake by analyzing meal images or may obtain them from information input by the patient via the operation receiver 22.

The controller 21 causes the display 23 to display the first medical information and note information (information corresponding to the patient who uses the patient terminal 20) that is stored in the patient information management server 10.

(Configuration of Doctor Terminal)

As shown in FIG. 1, the doctor terminal 30 includes a controller 31 (hardware processor), an operation receiver 32, a display 33, a communication unit 34, and a storage 35. These components are connected via a bus.

The controller 31, the display 33, the communication unit 34, and the storage 35 of the doctor terminal 30 are similar to the controller 21, the display 23, the communication unit 24, and the storage 25 of the patient terminal 20. Therefore, the description thereof is omitted.

The operation receiver 32 includes: a keyboard including cursor keys, character entry keys, and various function keys; and a pointing device, such as a mouse. The operation receiver 32 outputs operation signals input by the manipulation of the keyboard or the mouse to the controller 31.

When the doctor terminal 30 is a tablet, the operation receiver 32 includes operation keys such as a power key for turning on/off power; and a touchscreen layered on the display 33. The operation receiver 32 outputs, to the controller 31, operation signals corresponding to the respective operation keys and operation signals corresponding to positions touched by the finger of the operator.

The controller 31 obtains the first medical information that includes vital information of the patient and that is input by the patient terminal 20 used by the patient. More specifically, the controller 31 obtains, via the communication unit 34, the first medical information of the diagnosis target patient that is stored in the storage 13 of the patient information management server 10. The controller 31 thus functions as an obtaining unit.

The controller 31 displays the obtained first medical information on the display 33.

While displaying the first medical information on the display 33, the controller 31 receives inputs of note information to the first medical information and attaches the note information to the first medical information. The note information is input by a healthcare practitioner manipulating the operation receiver 32. The controller 31 thus functions as an attaching unit. For example, the controller 31 stores the note information in association with coordinates on the display screen of the first medical information. This clearly shows where the note information is located on the display window of the first medical information and which information the note information addresses.

The note information includes at least one among annotations, letters, symbols, comments, and audio data.

Annotations may be figures such as circles, triangles, and rectangles; or marks prepared beforehand. Annotations are not limited to specific ones.

Comments are information that describes contents to be conveyed from the healthcare practitioner to the patient.

Audio data describes contents to be conveyed from the healthcare practitioner to the patient by audio.

The controller 31 outputs the first medical information to which the note information is attached. The controller 31 thus functions as an outputting unit.

The controller 31 displays the first medical information to which the note information is attached on the display 33 (outputs display).

When the note information is attached to the first medical information, the controller 31 superposes the note information on the first medical information and displays both information.

The controller 31 attaches the note information to a first region in which the first medical information is displayed. In the case, the note information is displayed on the first medical information.

The controller 31 may attach the note information to a second region different from the first region in which the first medical information is displayed. In the case, the note information is displayed in a region different from the first medical information.

The correspondence between an item/portion of the first medical information and the note information may be indicated by the position of the note information or by a line that connects the note information to the corresponding position (region) of the first medical information.

The controller 31 outputs the first medical information attached with the note information to the patient terminal 20 of the patient corresponding to the first medical information (sends the first medical information to the outside). The outputting of the first medical information attached with the note information to the patient terminal 20 includes not only sending the first medical information directly to the patient terminal 20 from the doctor terminal 30 but also sending the first medical information to the patient information management server 10 so that the information can be referred to at the patient terminal 20 (indirect outputting).

(Operation of Medical Information Management System)>

Next, the operation of the medical information management system 100 is described.

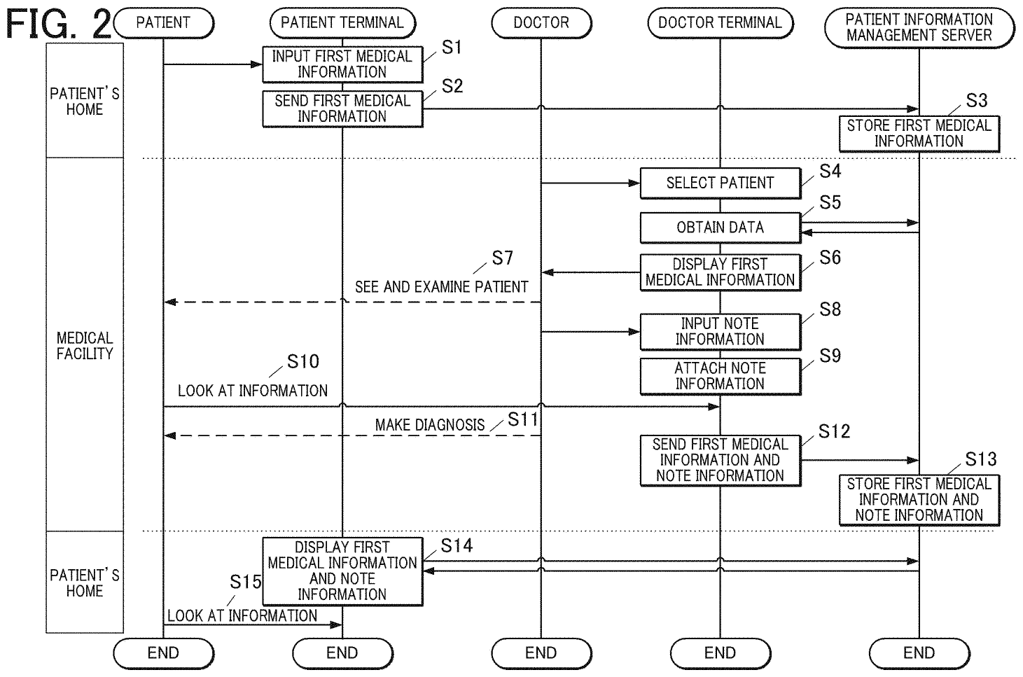

FIG. 2 shows the ladder chart of the process to be performed by the medical information management system 100. FIG. 2 shows the patient who manipulates the patient terminal 20 and the doctor who manipulates the doctor terminal 30, as well as the patient information management server 10, the patient terminal 20, and the doctor terminal 30.

The patient inputs first medical information including vital information by using the patient terminal 20 at home (Step S1). For example, the controller 21 of the patient terminal 20 obtains the patient's vital information from the vital measurement apparatus via the communication unit 24. The controller 21 obtains the number of steps and the traveled distance, which are calculated on the basis of walking of the patient and stored in the storage 25. The controller 21 also obtains information on the exercise done by the patient (e.g., the type, the number of times, the duration of the exercise), medication information, feeling information, and so forth input by the patient through the operation receiver 22. The controller 21 also obtains meal images (image data) generated by the camera mounted on the patient terminal 20. The first medical information is associated with the measurement date and time, input date and time, obtained date and time, and target date and time.

The controller 21 of the patient terminal 20 associates the first medical information with the patient information (e.g., patient ID) of the patient corresponding to the patient terminal 20 and sends the information to the patient information management server 10 via the communication unit 24 (Step S2).

The controller 11 of the patient information management server 10 receives the first medical information and the patient information from the patient terminal 20 via the communication unit 12 and stores the first medical information in association with the patient information in the storage 13 (Step S3).

Steps S1 to S3 are repeated every time the patient inputs the first medical information.

Next, the process moves to steps when the patient visits the medical facility.

At the doctor terminal 30, the doctor selects the diagnosis target patient by manipulating the operation receiver 32 while seeing the patient in the medical facility (Step S4).

The controller 31 of the doctor terminal 30 obtains data of the selected patient from the patient information management server 10 via the communication unit 34 (Step S5). More specifically, the controller 31 sends, via the communication unit 34, a request for the first medical information of the selected patient to the patient information management server 10.

When receiving the request for the first medical information of the patient selected by the doctor terminal 30 via the communication unit 12, the controller 11 of the patient information management server 10 reads the first medical information associated with the patient information of the selected patient in the storage 13 and sends the first medical information to the doctor terminal 30 via the communication unit 12.

The controller 31 of the doctor terminal 30 obtains, via the communication unit 34, the first medical information of the diagnosis target patient from the patient information management server 10.

The controller 31 of the doctor terminal 30 displays the first medical information of the diagnosis target patient (medical care guidance window) on the display 33 (Step S6).

The doctor sees the patient and asks the patient about his condition while referring to the first medical information displayed on the display 33 of the doctor terminal 30 (Step S7).

The doctor inputs note information with respect to the first medical information by operating the operation receiver 32 while showing the first medical information on the display 33 of the doctor terminal 30 to the patient (Step S8). For example, the doctor points out the part of the vital information that deviates from a reference range and inputs information to be attended by the patient. The controller 31 of the doctor terminal 30 receives selection of the type of note information (e.g., curve drawing, arrows, marks, comments) by the doctor manipulating the operation receiver 32 and receives specification of the position at which the note information is placed on the display screen of the first medical information on the display 33. When comments are selected, the controller 31 receives inputs of character strings.

The controller 31 of the doctor terminal 30 attaches the input note information to the first medical information (Step S9). More specifically, the controller 31 places the note information at the specified position (coordinates) on the display screen and generates image data of the medical care guidance window. The image data of the medical care guidance window includes a layer containing the first medical information and a layer containing the note information, wherein the note information layer is superposed on the first medical information layer. That is, the controller 31 superposes the note information on the first medical information and displays the note information and the first medical information.

The patient looks at the first medical information and the note information displayed on the display 33 of the doctor terminal 30 while receiving explanations from the doctor (Step S10).

The doctor diagnoses the patient (Step S11).

At the doctor terminal 30, when the doctor instructs saving of the medical care guidance window by manipulating the operation receiver 32, the controller 31 sends the first medical information and the note information (image data of the medical care guidance window) to the patient information management server 10 via the communication unit 34 (Step S12). The image data of the medical care guidance window includes the layer containing the first medical information and the layer containing the note information.

At the patient information management server 10, the controller 11 receives the first medical information and the patient information from the doctor terminal 30 via the communication unit 12, and stores, in the storage 13, the first medical information and the note information (image data of the medical care guidance window) in association with the patient information of the diagnosis target patient (Step S13).

The patient then returns home from the medical facility.

When the patient wants to check his/her medical information after returning home, the patient accesses the patient information management server 10 from the patient terminal 20. At the patient terminal 20, the patient specifies the predetermined URL for accessing the patient information management server 10 and inputs the patient ID and so forth on the log-in window by manipulating the operation receiver 22. The controller 21 obtains the image data of the medical care guidance window from the patient information management server 10 in accordance with the application program and displays the medical care guidance window on the display 23. The controller 11 of the patient information management server 10 obtains the image data of the medical care guidance window, which is associated with the patient's patient information (e.g., patient ID), from the storage 13, and provides the image data to the patient terminal 20 via the communication unit 12. The display 23 of the patient terminal 20 displays the first medical information and the note information of the patient who corresponds to the patient terminal 20 (step S14).

The patient looks at the first medical information and the note information displayed on the display 23 of the patient terminal 20 (Step S15).

Then the process ends.

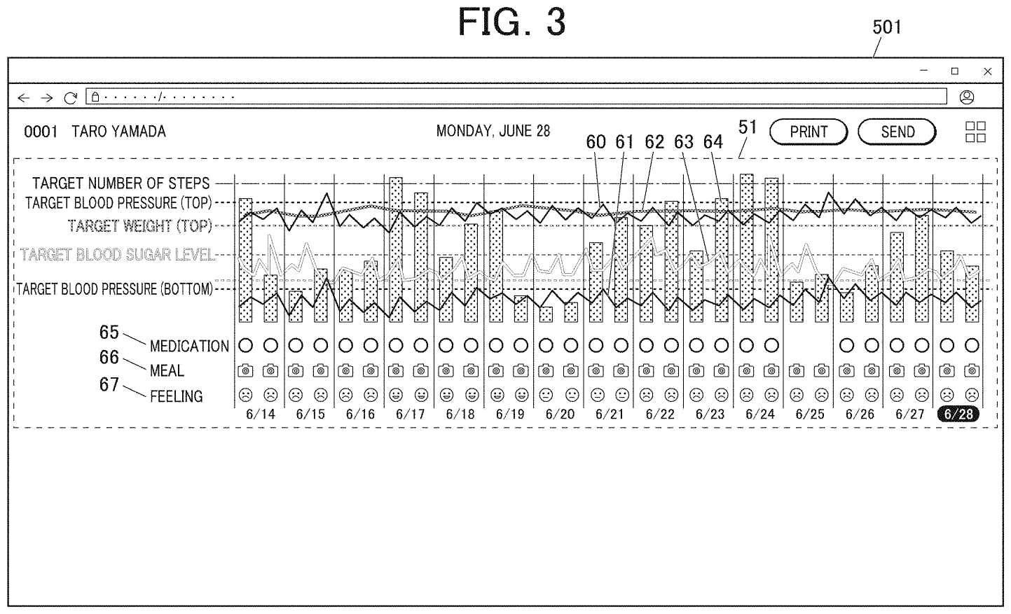

FIG. 3 shows an example of the medical care guidance window 501 displayed on the display 33 of the doctor terminal 30 in Step S6.

The medical care guidance window 501 includes the first medical information region 51.

The first medical information region 51 shows the first medical information.

The first medical information region 51 shows dates along the horizontal axis and shows line graphs indicating changes of the top blood pressure 60, the bottom blood pressure 61, the weight 62, and the blood sugar level 63. The first medical information region 51 also shows the column graph of the number of steps 64 that is measured for every half a day. The first medical information region 51 also has a medication section 65, a meal section 66, and a feeling section 67. The medication section 65 shows whether the patient took medicine at respective dosage timings (e.g., morning and evening) (circle: medicine taken, blank: medicine not taken). The meal section 66 shows camera marks at respective meal timings. When a camera mark of a meal is selected, the image of the meal is shown. The feeling section 67 shows marks indicating feelings at the respective input timings (good, normal, bad, etc.).

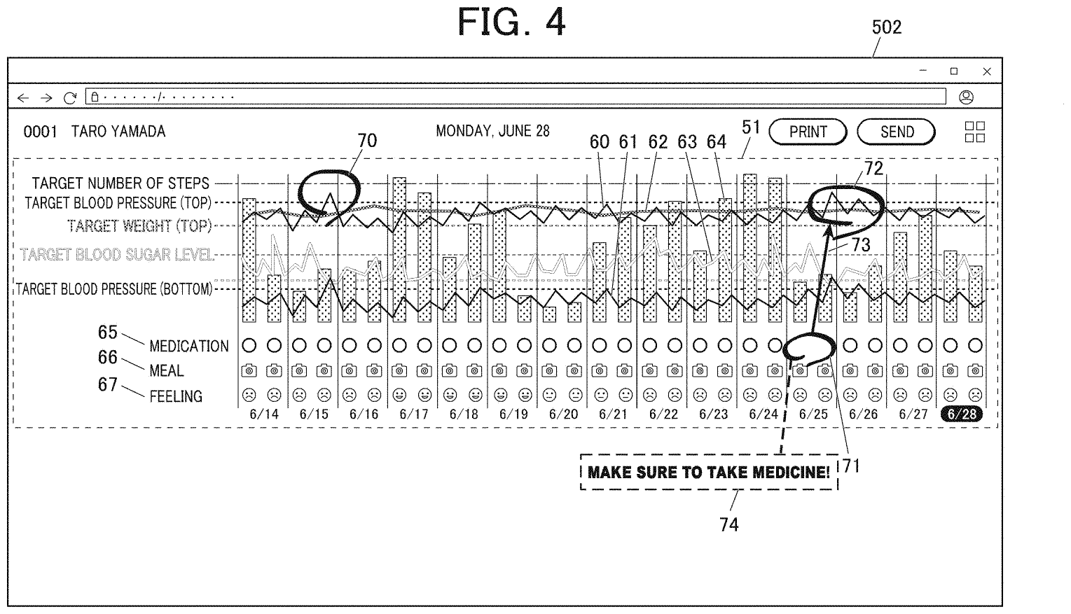

FIG. 4 shows an example of the medical care guidance window 502 to which the note information is attached in Step S9. Aspects of the medical care guidance window 502 that are the same as the medical care guidance window 501 in FIG. 3 are not described herein.

In the medical care guidance window 502, note information items 70 to 74 are attached.

The note information item 70 is an annotation (circle) for emphasizing that the top blood pressure 60 exceeds the target top blood pressure.

The note information item 71 is an annotation (circle) for emphasizing that June 25th has no medication record in the medication section 65.

The note information item 72 is an annotation (circle) for emphasizing that the top blood pressure 60 exceeds the target top blood pressure.

The note information item 73 is an arrow for indicating the correlation between the blood pressure and medication. Specifically, the note information item 73 indicates that the increase in blood pressure seems to be due to the failure to take medicine.

The note information item 74 is a comment of “make sure to take medicine!” to the patient. The note information item 74 is associated with the part where no medication is recorded in the medication section 65.

In the medical care guidance window 502, the note information items 70 to 73 are displayed in the first medical information region 51 (first region), and the note information item 74 is displayed in a region (second region) different from the first medical information region 51.

The user (doctor) can select whether or not to display the note information together with the first medical information on the medical care guidance window displayed on the display 33 of the doctor terminal 30, by manipulating the operation receiver 32. When the doctor selects displaying the note information, the controller 31 superposes the layer of the note information on the layer of the first medical information and displays these layers as the medical care guidance window. When the doctor selects not displaying the note information, the controller 31 does not superpose the layer of the note information and displays only the layer of the first medical information as the medical care guidance window.

The medical care guidance window 502 in FIG. 4 is also displayed on the display 23 of the patient terminal 20 when the patient looks at the medical care guidance window at home. On the patient terminal 20, the patient can select whether or not to display the note information by manipulating the operation receiver 22.

When the doctor at the doctor terminal 30 attaches audio data as the note information to the first medical information, a microphone mounted on (or connected to) the doctor terminal 30 obtains voices of the doctor. The doctor terminal 30 sends the audio data (note information) to the patient information management server 10 along with the image data of the medical care guidance window (first medical information) and the position of the audio data (note information) on the medical care guidance window specified by the doctor. At the patient information management server 10, the storage 13 stores the image data of the medical care guidance window, the position of the note information, and the audio data in association with the patient information of the diagnosis target patient.

When the patient at the patient terminal 20 refers to the first medical information in the patient information management server 10, the display 23 displays a speaker mark at the position specified by the doctor on the medical care guidance window, for example. When the patient presses the speaker mark by manipulating the operation receiver 22, the voice based on the audio data is reproduced.

As described above, according to the first embodiment, the controller 31 of the doctor terminal 30 attaches note information to the first medical information being displayed on the display 33 in accordance with the manipulation of the operation receiver 32 by the healthcare practitioner; and outputs the first medical information to which the note information is attached. Thus, the note information, which is input by the medical practitioner, can be presented so as to be easily understood in association with the first medical information, which is input by the patient, and which includes vital information.

Further, the controller 31 of the doctor terminal 30 displays the first medical information to which the note information is attached on the display 33. Thus, the note information can be displayed together with the first medical information.

Further, the controller 31 of the doctor terminal 30 generates image data of the medical care guidance window that includes the layer of the first medical information and the layer of the note information, the layers being separated from each other. Thus, the note information can be superposed on the first medical information for display.

Further, whether or not to display the note information on the first medical information can be switched.

Further, the controller 31 of the doctor terminal 30 outputs the first medical information to which the note information is attached to the patient information management server 10 such that the information can be referred to from the patient terminal 20. (i.e., the controller 31 indirectly outputs the first medical information attached with the note information to the patient terminal 20). This allows the patient to look at the first medical information and the note information on the patient terminal 20. The patient can easily grasp the doctor's advice regarding the first medical information (e.g., vital information) input by the patient. This improves viewability.

Further, the controller 31 attaches the note information to a first region in which the first medical information is displayed (e.g., the first medical information region 51 in FIG. 4). This clearly shows the correspondence between the note information and an item/portion of the first medical information.

Further, the controller 31 attaches the note information to a second region that is different from the first region in which the first medical information is displayed. Thus, the note information can be presented without obstructing the display of the first medical information.

### Second Embodiment

(Configuration of Medical Information Management System)

Next, a second embodiment of the present invention is described.

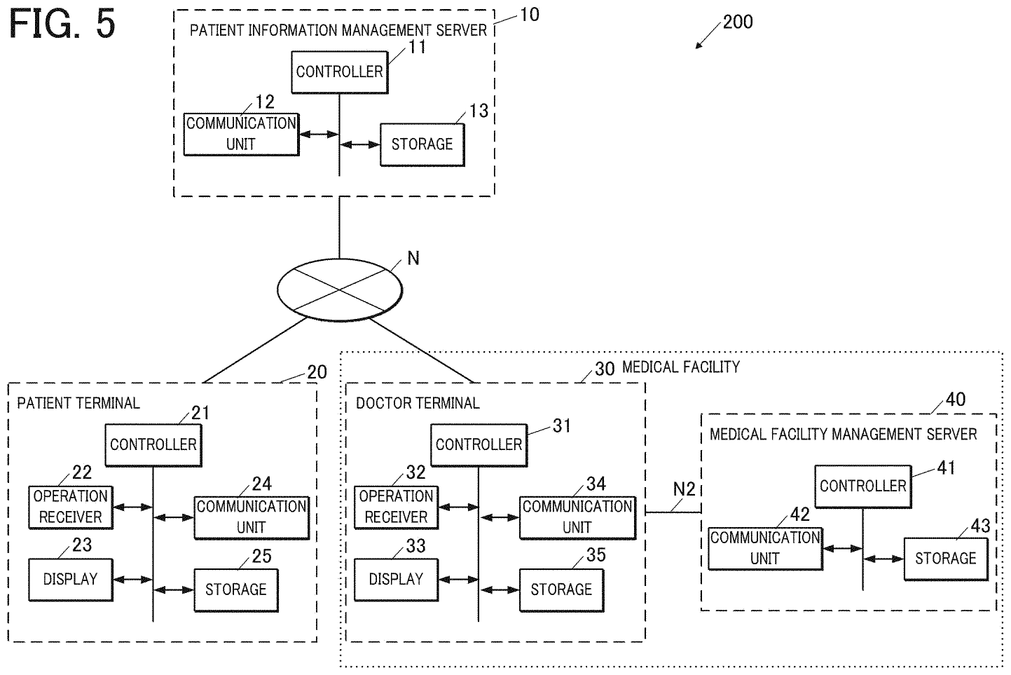

FIG. 5 shows a structure of a medical information management system 200 in a second embodiment. As shown in FIG. 5, the medical information management system 200 includes the patient information management server 10; the patient terminal 20 to be used by a patient; the doctor terminal 30 (medical information management apparatus) to be used by a doctor in a medical facility; and a medical facility management server 40 that manages information inside the medical facility. The patient information management server 10, the patient terminal 20, and the doctor terminal 30 are connected to exchange data over a communication network N (e.g., internet). The doctor terminal 30 and the medical facility management server 40 are connected over a communication network N2 (e.g., local area network (LAN) inside the medical facility) to exchange data.

Aspects of the medical information management system 200 that are the same as those of the medical information management system 100 in the first embodiment are denoted by the same reference numerals, and the description thereof is omitted. The configuration and processes specific to the second embodiment are described below.

The doctor terminal 30 obtains (i) first medical information (e.g., vital information) input by the patient terminal 20, (ii) second medical information (e.g., examination data) from an apparatus inside the medical facility (medical facility management server 40), and (iii) third medical information (e.g., reference information, comment information).

The doctor terminal 30 can attach note information (e.g., annotation) not only to the first medical information but also to the second and third medical information.

(Configuration of Medical Facility Management Server)

As shown in FIG. 5, the medical facility management server 40 includes a controller 41, a communication unit 42, and a storage 43. These components are connected via a bus.

The controller 41 includes a CPU and a RAM and centrally controls processing operations of the components of the medical facility management server 40. More specifically, the CPU of the controller 41 reads various processing programs stored in the storage 43, loads them into the RAM, and performs various processes in cooperation with the programs.

The communication unit 42 includes a network interface. The communication unit 42 sends and receives data to and from external apparatuses (e.g., doctor terminal 30) connected over the communication network N2.

The storage 43 consists of a HDD and/or a SSD, for example. The storage 43 stores various processing programs and parameters and files necessary for executing the programs.

The storage 43 stores the second medical information obtained from examinations on the patient (e.g., examination data) in association with the patient information. The second medical information includes examination data, such as (i) medical images generated in image examinations by modalities (e.g., ultrasound imaging, X-ray imaging, computed tomography (CT), magnetic resonance imaging (MM), scintigraphy) and (ii) measured values obtained from examinations (e.g., electrocardiogram, spirometer).

(Configuration of Doctor Terminal)

The controller 31 (obtaining unit) of the doctor terminal 30 obtains the second medical information obtained from examinations of the patient. More specifically, the controller 31 obtains, via the communication unit 34, the second medical information of the diagnosis target patient that is stored in the storage 43 of the medical facility management server 40.

The controller 31 (attaching unit) attaches note information (second note information) to the second medical information in accordance with the manipulation of the operation receiver 32 by healthcare practitioner.

The controller 31 (outputting unit) outputs first combination information that is the combination of at least the first medical information to which note information is attached and the second medical information. In the first combination information, the second medical information may be attached with note information.

The controller 31 (obtaining unit) obtains the third medical information (e.g., reference information, comment information) that is related to the first medical information. The third medical information includes reference information to be referred to in examining the first medical information (e.g., general materials) and comment information that is freely input by the healthcare practitioner with respect to the first medical information or to the diagnosis target patient.

For example, the controller 31 obtains the third medical information selected by the healthcare practitioner manipulating the operation receiver 32. The controller 31 may obtain the third medical information stored in an external apparatus via the communication unit 34 or may obtain the third medical information stored in the storage 35.

The controller 31 also obtains the third medical information input by the healthcare practitioner manipulating the operation receiver 32.

The controller 31 (attaching unit) attaches note information (third note information) to the third medical information according to the manipulation of the operation receiver 32 by the healthcare practitioner.

The controller 31 (outputting unit) outputs second combination information that is the combination of at least the first medical information to which note information is attached and the third medical information. In the second combination information, the third medical information may be attached with note information.

The note information attached to the second medical information and the note information attached to the third medical information include at least one among annotations, characters, symbols, comments, and audio data, as with the note information attached to the first medical information.

(Operation of Medical Information Management System)

Next, the operation of the medical information management system 200 is described.

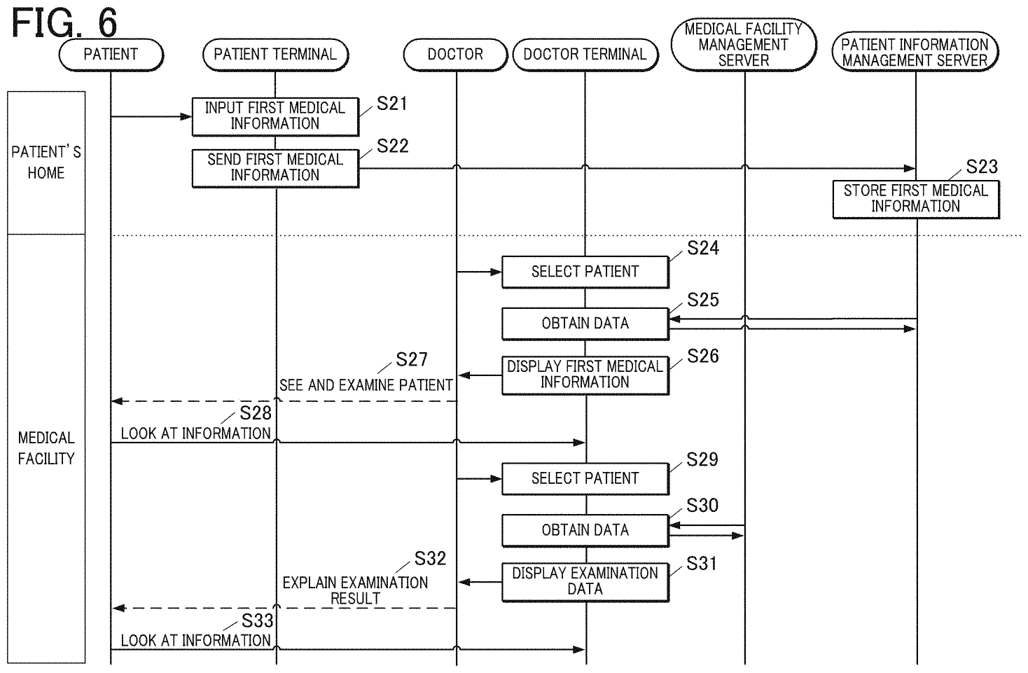

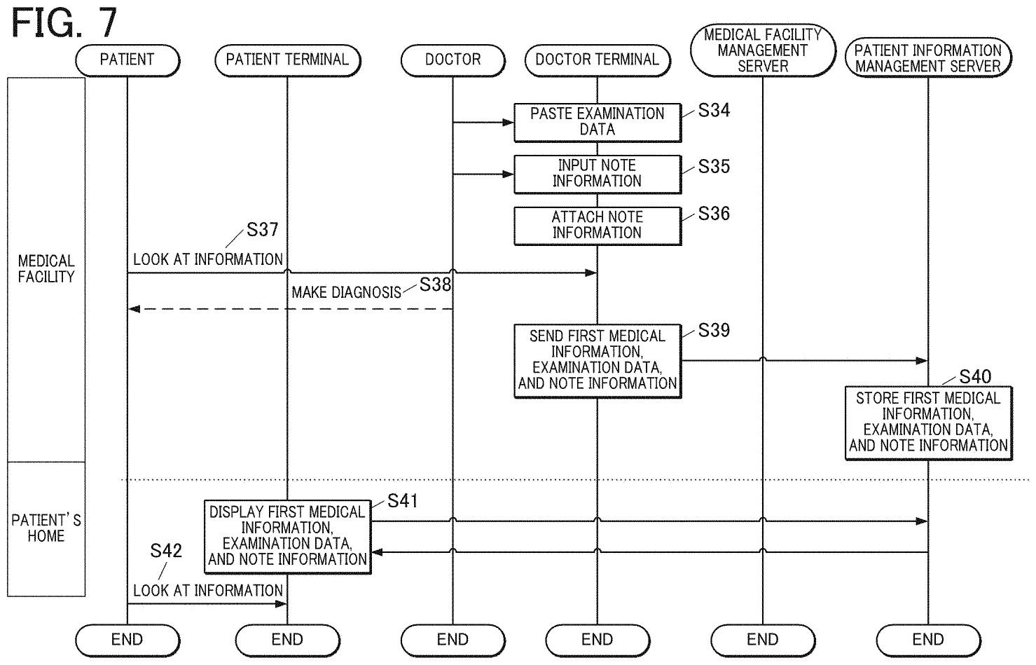

FIG. 6 and FIG. 7 show the ladder chart of the process to be performed by the medical information management system 200. FIG. 6 and FIG. 7 show the patient who manipulates the patient terminal 20 and the doctor who manipulates the doctor terminal 30, as well as the patient information management server 10, the patient terminal 20, the doctor terminal 30, and the medical facility management server 40.

In Steps S21 to S23, the patient registers the first medical information (e.g., vital information) to the patient information management server 10 by using the patient terminal 20 at home. Steps S21 to S23 are the same as Steps S1 to S3 in FIG. 2 and are therefore not described.

Steps S24 to S27 are performed after the patient visits the medical facility. Steps S24 to S27 are the same as Steps S4 to S7 in FIG. 2 and are therefore not described.

While receiving explanations from the doctor, the patient looks at the first medical information displayed on the display 33 of the doctor terminal 30 (Step S28).

At the doctor terminal 30, the doctor selects the diagnosis target patient by manipulating the operation receiver 32 to use information stored in the medical facility (medical facility management server 40) (Step S29).

The controller 31 of the doctor terminal 30 obtains data of the selected patient from the medical facility management server 40 via the communication unit 34 (Step S30). Herein, the controller 30 may obtain only an examination data item that is selected by the doctor from among the examination data items stored in the medical facility management server 40. The controller 31 sends a request for examination data of the selected patient to the medical facility management server 40 via the communication unit 34.

When receiving the request for the examination data of the selected patient from the doctor terminal 30 via the communication unit 42, the controller 41 of the medical facility management server 40 reads out examination data associated with patient information of the selected patient from the storage 43 and sends the examination data to the doctor terminal 30 via the communication unit 42.

The controller 31 of the doctor terminal 30 receives the examination data of the diagnosis target patient from the medical facility management server 40 via the communication unit 34.

The controller 31 of the doctor terminal 30 displays the examination data of the diagnosis target patient on the display 33 (Step S31).

The doctor explains the result of the examination to the patient while referring to the examination data displayed on the display 33 of the doctor terminal 30 (Step S32).

The patient looks at the examination data displayed on the display 33 of the doctor terminal 30 while receiving explanations from the doctor (Step S33).

The process proceeds to FIG. 7. The doctor at the doctor terminal 30 manipulates the operation receiver 32 to paste the examination data onto the display screen of the first medical information on the display 33 (Step S34). More specifically, when the doctor specifies the position to which the examination data is pasted on the display screen by manipulating the operation receiver 32, the examination data is displayed on the specified position.

The doctor also inputs note information to the first medical information and/or the examination data by manipulating the operation receiver 32 (Step S35).

The controller 31 of the doctor terminal 30 attaches the input note information to the first medical information and/or the examination information (Step S36). More specifically, the controller 31 places the note information at the specified position (coordinates) on the display screen and generates image data of the medical care guidance window. The image data of the medical care guidance window includes (i) a layer containing the first medical information and the examination data and (ii) a layer containing the note information and being superposed on the layer containing the first medical information and the examination data. The image data of the medical care guidance window that includes the first medical information, examination data, and note information to the first medical information corresponds to the first combination information that is the combination of at least the first medical information to which the note information is attached and the second medical information.

The patient looks at the first medical information, the examination data, and the note information displayed on the display 33 of the doctor terminal 30, while receiving explanations from the doctor (Step S37).

The doctor diagnoses the patient (Step S38).

When the doctor at the doctor terminal 30 instructs saving of the medical care guidance window by manipulating the operation receiver 32, the controller 31 sends the first medical information, the examination data, and the note information (image data of the medical care guidance window) to the patient information management server 10 via the communication unit 34 (Step S39). The image data of the medical care guidance window includes (i) the layer containing the first medical information and the examination data and (ii) the layer containing the note information.

The controller 11 of the patient information management server 10 receives the first medical information, the examination data, and the note information the doctor terminal 30 via the communication unit 12. The controller 11 stores the first medical information, the examination data, and the note information (image data of the medical care guidance window) in association with the patient information of the diagnosis target patient in the storage 13 (Step S40).

The patient then returns home from the medical facility.

When the patient wants to check his/her medical information after returning home, the patient accesses the patient information management server 10 from the patient terminal 20. At the patient terminal 20, the patient specifies the predetermined URL for accessing the patient information management server 10 and inputs the patient ID and so forth on the log-in window by manipulating the operation receiver 22. The controller 21 obtains the image data of the medical care guidance window from the patient information management server 10 in accordance with the application program and displays the medical care guidance window on the display 23. The controller 11 of the patient information management server 10 obtains the image data of the medical care guidance window, which is associated with the patient's patient information (e.g., patient ID), from the storage 13, and provides the image data to the patient terminal 20 via the communication unit 12. The display 23 of the patient terminal 20 displays the first medical information, the examination data, and the note information of the patient who corresponds to the patient terminal 20 (Step S41).

The patient looks at the first medical information, the examination data, and the note information displayed on the display 23 of the patient terminal 20 (Step S42).

Then the process ends.

In the process shown in FIG. 6 and FIG. 7, the medical care guidance window includes the first medical information and the examination data (second medical information). The medical care guidance window may further include the third medical information. The image data of the medical care guidance window that includes the first medical information, third medical information, and note information to the first medical information corresponds to the second combination information that is the combination of at least the first medical information to which the note information is attached and the third medical information. The medical care guidance window may include the first medical information and third medical information without including the second medical information.

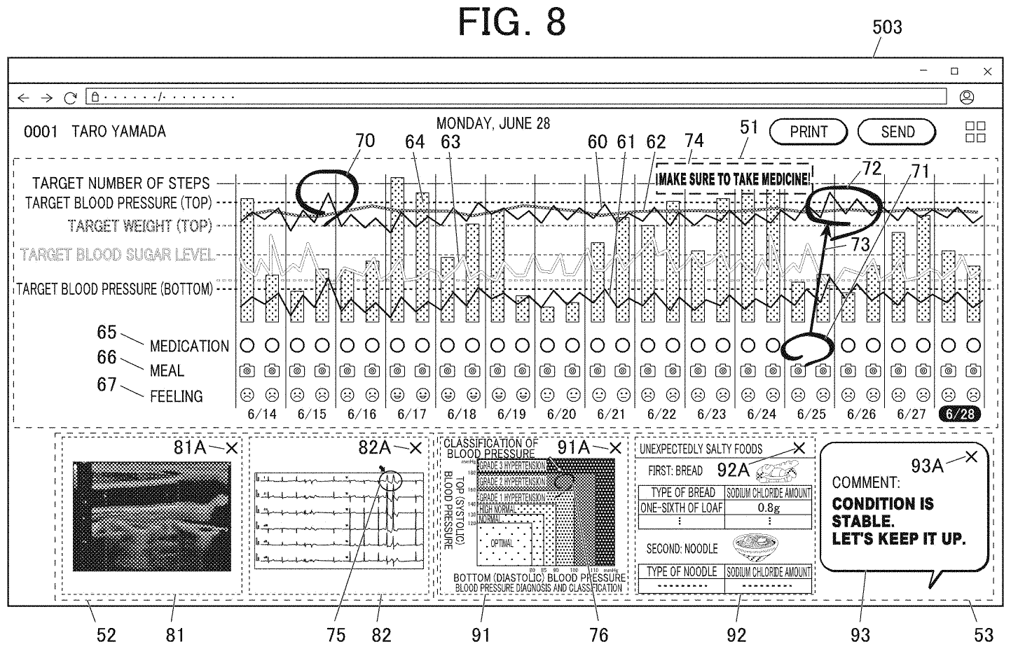

FIG. 8 shows an example of the medical care guidance window 503 in which note information is attached. The medical care guidance window 503 is displayed on the display 33 of the doctor terminal 30.

The medical care guidance window 503 includes the first medical information region 51, the second medical information region 52, and the third medical information region 53. Herein, the format of the medical care guidance window 503 is prepared beforehand. The doctor pastes medical information onto the second medical information region 52 and the third medical information region 53 while explaining to the patient.

The first medical information region 51 is the same as that in the medical care guidance window 501 shown in FIG. 3.

The second medical information region 52 is for displaying the second medical information. The second medical information region 52 is blank until the doctor selects the second medical information. For example, in Step S34, the doctor manipulates the operation receiver 32 to drag and drop the examination data displayed on the display 33 of the doctor terminal 30 onto the second medical information region 52 in the medical care guidance window 503. Thus, the examination data selected by the doctor is pasted to the second medical information region 52.

In FIG. 8, the second medical information region 52 includes an ultrasound image 81 and electrocardiogram data 82.

The ultrasound image 81 is a medical image obtained by performing an ultrasonography to the carotid artery of the patient. The ultrasound image 81 is used in examining the obstruction of a blood vessel.

The electrocardiogram data 82 is the measurement result of an electrocardiography to the patient. An abnormal part of the electrocardiogram data 82 is attached with note information item 75. The note information item 75 is a line enclosing the part of two consecutive times of premature ventricular contractions and an arrow on the measurement data in the electrocardiogram.

The third medical information region 53 is for displaying the third medical information. The third medical information region 53 is blank until the doctor selects or inputs the third medical information.

In FIG. 8, the third medical information region 53 includes a blood pressure classification diagram 91, foodstuff information 92, and a comment 93.

The blood pressure classification diagram 91 is a material for explaining the state of the blood pressure values of the patient based on the combination of the top blood pressure and the bottom blood pressure. Part of the blood pressure classification diagram 91 corresponding to the patient's blood pressure values (measured values) is attached with a note information item 76. The note information item 76 indicates, with an arrow, the part corresponding to the combination of the top blood pressure and bottom blood pressure of the patient in the blood pressure classification diagram 91.

The foodstuff information 92 is a material for explaining unexpectedly salty foods.

The comment 93 is the doctor's comment to the patient.

As the second medical information to be shown in the second medical information region 52 and the third medical information to be shown in the third medical information region 53, medical information may be automatically extracted, such as (i) medical information corresponding to vital information of the diagnosis target patient and (ii) medical information corresponding to tendency/changes of the vital information. Examples of the medical information corresponding to vital information include an image of blood vessels of the diagnosis target patient, if the patient has a high blood pressure. Examples of the medical information corresponding to the tendency/changes of vital information include appropriate medical information, such as data of an expected disease, on the basis of whether the blood pressure has increased rapidly or whether the blood pressure continues to be at a high level.

The image data of the medical care guidance window, which is generated by the doctor terminal 30, includes (i) a layer containing medical information (first, second, and third medical information) and (ii) a layer containing note information.

The user (doctor) can select whether or not to display note information together with medical information (first, second, and third medical information) on the medical care guidance window displayed on the display 33 of the doctor terminal 30 by manipulating the operation receiver 32. When the user selects displaying note information, the controller 31 superposes the layer of note information on the layer of medical information to display the medical care guidance window. When the doctor selects not displaying the note information, the controller 31 displays only the layer of medical information as the medical care guidance window, without superposing the layer of note information.

The ultrasound image 81, the electrocardiogram data 82, the blood pressure classification diagram 91, the foodstuff information 92, and the comment 93 are provided with delete buttons 81A, 82A, 91A, 92A, and 93A, respectively. The user (doctor) can delete the ultrasound image 81, the electrocardiogram data 82, the blood pressure classification diagram 91, the foodstuff information 92, and the comment 93 by pressing the delete buttons 81A, 82A, 91A, 92A, and 93A.

Herein, items of the second medical information and third medical information can be deleted. In addition, individual items of the first medical information may also be deletable.

Image data (data for displaying information) corresponding to individual data items of the second medical information shown in the second medical information region 52 and the third medical information shown in the third medical information region 53 may also be sent to the patient information management server 10 from the doctor terminal 30 and stored in the storage 13.

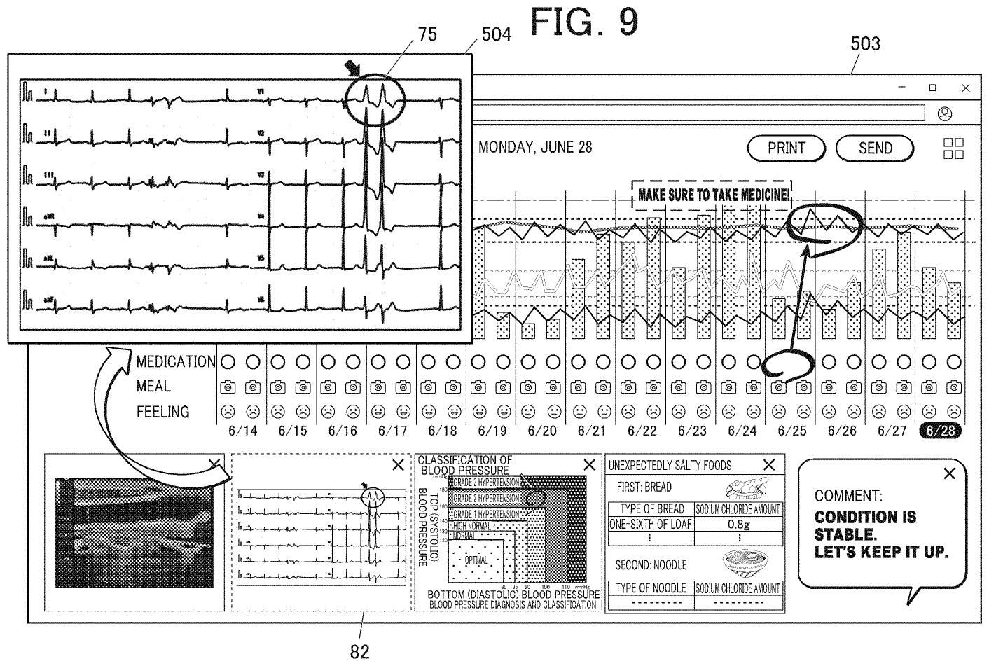

For example, when the doctor at the doctor terminal 30 specifies the electrocardiogram data 82 in the second medical information region 52 of the medical care guidance window 503 by manipulating the operation receiver 32, an electrocardiogram window 504 is displayed, as shown in FIG. 9. In the electrocardiogram window 504, the electrocardiogram data 82 is enlarged. In order to show the electrocardiogram window 504, the storage 13 of the patient information management server 10 stores not only the image data of the medical care guidance window 503 but also the image data of the electrocardiogram data only.

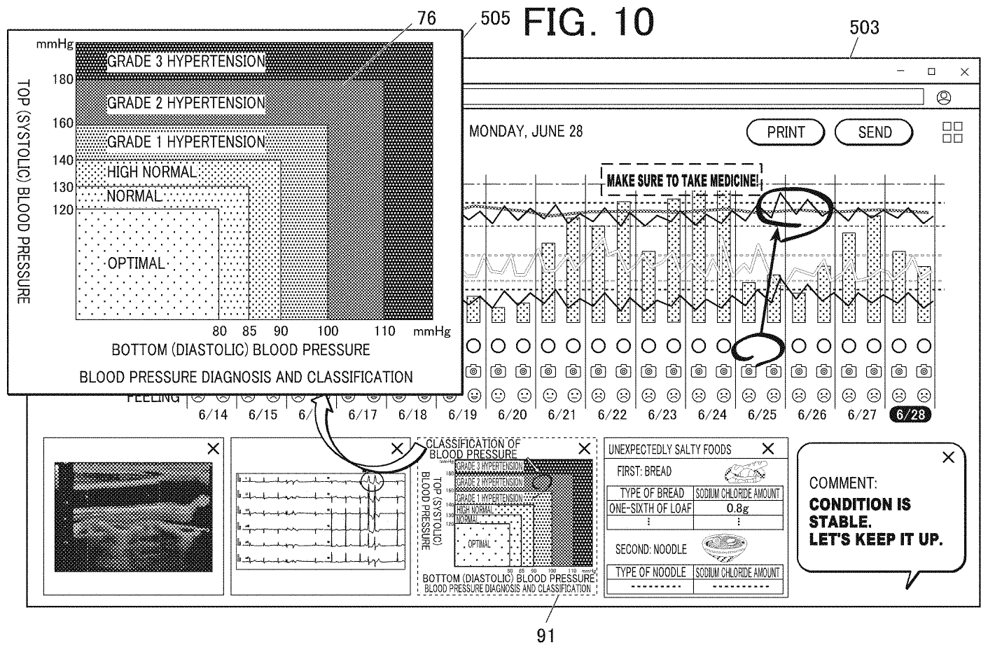

When the doctor at the doctor terminal 30 specifies the blood pressure classification diagram 91 in the third medical information region 53 on the medical care guidance window 503 by manipulating the operation receiver 32, the blood pressure classification diagram window 505 showing the enlarged blood pressure classification diagram 91 is displayed, as shown in FIG. 10. Thus, in order to show the blood pressure classification diagram window 505, the storage 13 of the patient information management server 10 stores not only the image data of the medical care guidance window 503 but also the image data of the blood pressure classification diagram table alone.

The medical care guidance window 502 in FIG. 8 is also displayed on the display 23 of the patient terminal 20 when the patient looks at the medical care guidance window at home. On the patient terminal 20, the patient can select whether or not to display the note information by manipulating the operation receiver 22. As shown in FIG. 9 and FIG. 10, the patient terminal 20 can also display enlarged second and third medical information when the second/third medical information is specified in the medical care guidance window.

As described above, according to the second embodiment, the controller 31 of the doctor terminal 30 attaches note information to the first medical information being displayed on the display 33 in accordance with the manipulation of the operation receiver 32 by the healthcare practitioner; and outputs the first medical information to which the note information is attached. Thus, the note information, which is input by the medical practitioner, can be presented so as to be easily understood in association with the first medical information, which is input by the patient, and which includes vital information.

Further, when, at the doctor terminal 30, the second medical information is pasted on the display screen of the first medical information and when note information is attached to the first medical information, the doctor terminal 30 can output first combination information that is the combination of at least the first medical information to which the note information is attached and the second medical information (image data of the medical care guidance window containing the first medical information to which the note information is attached and the second medical information).

Further, at the doctor terminal 30, when the third medical information is pasted on the display screen of the first medical information and when note information is attached to the first medical information at the doctor terminal 30, the doctor terminal 30 can output second combination information that is the combination of at least the first medical information to which the note information is attached and the third medical information (image data of the medical care guidance window containing the first medical information to which the note information is attached and the third medical information).

Further, the controller 31 of the doctor terminal 30 displays medical information (first medical information, second medical information, third medical information) to which note information is attached on the display 33. Thus, the controller 31 can present the note information together with the medical information.

Further, the controller 31 of the doctor terminal 30 generates image data of the medical care guidance window that includes the layer of medical information and the layer of note information, the layers being separated from each other. Thus, the note information can be superposed on the medical information for display.

Further, whether or not to display the note information on the medical information can be selected (switched).

Further, the controller 31 of the doctor terminal 30 outputs, to the patient information management server 10, the medical information to which the note information attached such that the information can be referred to from the patient terminal 20. That is, the controller 31 indirectly outputs the medical information to which the note information is attached to the patient terminal 20. This allows the patient to look at the medical information and the note information on the patient terminal 20.

In the medical care guidance window displayed on the display 33 of the doctor terminal 30, it may be determined whether the patient information (e.g., patient information included in the header of medical information) associated with the second medical information selected by the doctor as a pasting target matches with the patient information associated with the first medical information. When they do not match, the result of determination (alarm) may be presented.

When the doctor selects a medical image to paste, the display 33 of the doctor terminal 30 may display only the medical images that have already been interpreted (that can be used as the result of diagnosis) as candidate medical images.

Further, when the doctor selects a medical image to paste on the doctor terminal 30, it may be determined whether the selected medical image has already been interpreted. The pasting of the image may be allowed only when the image has already been interpreted.

The embodiments described above are examples of the medical information management apparatus according to the present invention and are not intended to limit the present invention. The detailed configuration and detailed operation of the components constituting the system can be appropriately modified without departing from the scope of the present invention.

For example, in the above embodiments, the patient information management server 10 stores the first medical information input at the patient terminal 20, and the controller 31 (obtaining unit) of the doctor terminal 30 (medical information management apparatus) obtains the first medical information from the patient information management server 10. However, the controller 31 of the doctor terminal 30 may obtain the first medical information directly from the patient terminal 20.

The doctor terminal 30 may also have the function of the patient information management server 10.

In the above embodiments, the patient information management server 10 stores medical information (first, second, and third medical information) to which note information is attached at the doctor terminal 30, and the patient at the patient terminal 20 refers to the medical information attached with the note information that is stored in the patient information management server 10. However, the medical information attached with the note information may be sent directly from the doctor terminal 30 to the patient terminal 20. Alternatively, the controller 21 of the patient terminal 20 may directly access the doctor terminal 30 to obtain the medical information to which the note information is attached.

In the above embodiments, the note information is associated with a specific position of the medical information by storing the note information in association with coordinates on the display screen of the medical information, as an example. As another example, the note information may be stored in association with the date or the day of the week of the first medical information (e.g., vital information). For example, the position of annotation (e.g., circle) is kept unchanged while the display range is changed by turning pages of displayed data, so that the present data can be compared with data on the same day of the last month or data on the same day of the last week.

The final output destination of at least the first medical information (e.g., vital information) and note information may not be the patient terminal 20.

For example, medical information and note information may be displayed on the terminal used by an imager. This allows the doctor to share information with the imager and allows the imager to pay attention to the information in the next imaging.

The medical information and note information may be displayed on the terminal used by a nurse. This allows the nurse to grasp the status of the patient.

According to the above embodiment, medical information, which includes at least first medical information and may include second and third medical information, can be separated from note information by using different layers, and the note information is superposed on the medical information for display. However, the note information may be embedded in the medical information. In the case, when the note information is attached to the medical information, the controller 31 of the doctor terminal 30 embeds the note information into the medical information to generate image data of the medical care guidance window. More specifically, the controller 31 generates image data of the medical information attached with the note information in the format of portable document format (PDF) or portable network graphics (PNG), for example. Such image data of the medical care guidance window is associated with patient information and stored in the storage 13 of the patient information management server 10. Thus, the medical care guidance window can be referred to from the patient terminal 20.

The program for executing various processes in the respective apparatuses may be stored in a portable storage medium(s). Further, a carrier wave can be used as a medium to provide data of the programs via a communication line.

1. A medical information management apparatus comprising a hardware processor that:
obtains first medical information that is input through a patient terminal used by a patient and that includes vital information of the patient;
attaches note information to the first medical information being displayed on a display in accordance with a manipulation of an operation receiver by a healthcare practitioner; and
outputs the first medical information to which the note information is attached.

2. The medical information management apparatus according to claim 1, wherein
the hardware processor displays the first medical information to which the note information is attached on the display.

3. The medical information management apparatus according to claim 2, wherein
when the note information is attached to the first medical information, the hardware processor superposes the note information on the first medical information and displays the first medical information and the note information.

4. The medical information management apparatus according to claim 2, wherein
when the note information is attached to the first medical information, the hardware processor embeds the note information in the first medical information and displays the first medical information and the note information.

5. The medical information management apparatus according to claim 1, wherein
the hardware processor outputs the first medical information to which the note information is attached to the patient terminal.

6. The medical information management apparatus according to claim 1, wherein
the hardware processor attaches the note information to a first region in which the first medical information is displayed.

7. The medical information management apparatus according to claim 1, wherein
the hardware processor attaches the note information to a second region that is different from a first region in which the first medical information is displayed.

8. The medical information management apparatus according to claim 1, wherein
the note information includes at least one among an annotation, a letter, a symbol, a comment, and audio data.

9. The medical information management apparatus according to claim 1, wherein
the hardware processor further obtains second medical information that is obtained in an examination of the patient, and
the hardware processor outputs first combination information that is a combination of at least the first medical information to which the note information is attached and the second medical information.

10. The medical information management apparatus according to claim 9, wherein
the hardware processor attaches second note information to the second medical information in accordance with a manipulation of the operation receiver by the healthcare practitioner.

11. The medical information management apparatus according to claim 1, wherein
the hardware processor further obtains third medical information that is related to the first medical information, and
the hardware processor outputs second combination information that is a combination of at least the first medical information to which the note information is attached and the third medical information.

12. The medical information management apparatus according to claim 11, wherein
the hardware processor attaches third note information to the third medical information in accordance with a manipulation of the operation receiver by the healthcare practitioner.

13. A non-transitory computer-readable storage medium storing a program that causes a computer to perform:
obtaining first medical information that is input through a patient terminal used by a patient and that includes vital information of the patient;
attaching note information to the first medical information being displayed on a display in accordance with a manipulation of an operation receiver by a healthcare practitioner; and
outputting the first medical information to which the note information is attached.
